(19) United States
(12) Patent Application Publication
(10) Pub. No.: US 2013/0255697 A1
(43) Pub. Date: Oct. 3, 2013

(54) Protective Sleep Mask

(71) Applicant: Barbara S. Thompson, Downers Grove, IL (US)

(72) Inventor: Barbara S. Thompson, Downers Grove, IL (US)

(21) Appl. No.: 13/433,947

(22) Filed: Mar. 29, 2012

(51) Int. Cl. A61F 9/04; A61M 16/06; A61F 11/06

(52) U.S. Cl. 128/858; 2/15; 2/423

(57) ABSTRACT

A protective mask for use with a CPAP system is disclosed. The protective mask aligns with a user's eye area to protect a user's eyes from any flow of air which may escape the air-delivery mask of the CPAP system. The composition of the protective mask is such that it is impermeable to air on one side and has a soft and non-irritating surface on the other side. The protective mask is preferably adjustable to accommodate various sized users and may include ear protectors to help facilitate uninterrupted sleep.

### TECHNICAL FIELD OF THE INVENTION

The present device relates to a sleep mask. Particularly, the present device relates to a sleep mask for CPAP patients.

### BACKGROUND OF THE INVENTION

Sleep apnea is a condition whereby a person ceases breathing for a period of time during sleep. The breathing lapses can leave the person's brain and body oxygen deprived. The condition can be life-threatening and even in the best of cases, it can be debilitating to an individual due to a lack of quality sleep.

Continuous positive airway pressure, or CPAP, is a relatively new form of technology developed to address the problem of sleep apnea. Various CPAP supplies have been developed including plastic face masks and nasal “pillows” to direct forced air into the nasal area of a sleeping person. The use of a facial mask system is probably the most common device for providing the continuous flow of air. The CPAP mask consists of a plastic, cupped breathing mask which is strapped in some manner to the person's head and includes a hose attached to the mask and leading to an air pump. The breathing mask fits over a user's nose and may include a soft, pliable outer edge to seal against the user's face to minimize escaping air. The continuous air pressure forces air into the person's lungs to counter the breathing cessation.

A significant problem with current CPAP systems, as shown in FIG. 1, is that air does escape from the mask and is regularly forced into the eyes of the sleeper. The air may cause eye dryness, eye irritation and may lead to other physical damage to the eye and an area around the eye over time.

The present invention is designed to address this issue by providing protection for a CPAP user's eye area.

### SUMMARY OF THE INVENTION

There is disclosed herein an improved protective sleep mask which avoids the disadvantages of prior devices while affording additional structural and operating advantages.

Accordingly, an eye-protection device for use with a CPAP machine and headgear, including an oxygen mask which covers a user's nose, is comprised of a first eye portion sized to extend from a person's eyebrow to an area below a person's cheekbone when in use, a second eye portion connected to the first eye portion and sized to extend from a person's eyebrow to an area below a person's cheekbone when in use, and a securing strap connected to each of the first and second eye portions for retaining the device on a user. The first eye portion and the second eye portion are comprised of a material having a soft porous inner surface layer and a non-porous outer surface layer.

In an embodiment of the protective mask, an edging of soft porous material, such as cotton, is applied along a periphery of the first eye portion and the second eye portion. Further, to provide greater utility, the securing strap has an adjustable length. Such adjustability may be provided through the use of a hook-and-loop connection, or other known means.

Optionally, the protective mask may be provided with ear protectors attached to the securing strap, positioned to cover a person's ears when in use. In such an embodiment, the position of the first and second ear protectors may be adjustable along a length of the securing strap.

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1 illustrates a typically CPAP mask system showing the proximity and vulnerability of the eyes to escaping air;

FIG. 2 is a front perspective view of an embodiment of the present protective mask;

FIG. 3 is a rear perspective view of the embodiment of FIG. 2;

FIG. 4 is a side view of an alternate embodiment of the present protective mask; and

FIG. 5 is a side view of another alternate embodiment of the present protective mask.

### DETAILED DESCRIPTION OF PREFERRED EMBODIMENTS

While this invention is susceptible of embodiments in many different forms, there is shown in the drawings and will herein be described in detail a preferred embodiment of the invention with the understanding that the present disclosure is to be considered as an exemplification of the principles of the invention and is not intended to limit the broad aspect of the invention to embodiments illustrated.

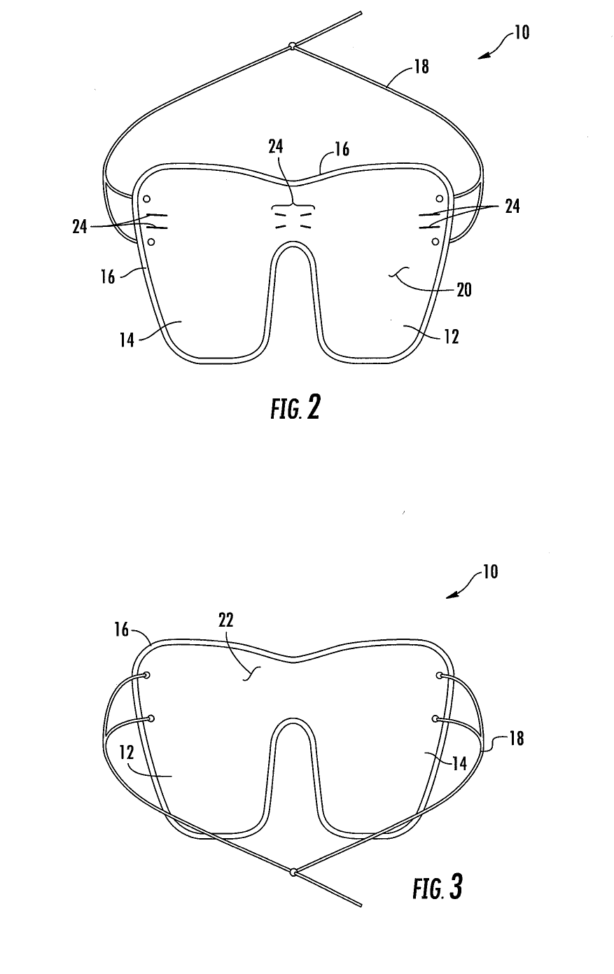

Referring to FIGS. 2-5, there is illustrated a protective mask, generally designated by the numeral 10. The mask 10 has a first and second eye portions, 12 and 14, connected together and a securing strap 18 connected to each of the eye portions, 12 and 14.

The body of the first and second eye portions, 12 and 14, are preferably comprised of a pliable vinyl material on the outer surface 20 with a soft inner surface 22, such as a cotton material. Vinyl material is non-porous and serves to block air-flow, while the soft inner surface 22 is comfortable and will not irritate the contacted skin of the user. The edge 16 of each eye portion, 12 and 14, is preferably comprised of a soft, cotton material as well. This edging 16 will help avoid any chafing as a result of the vinyl material contacting the user's facial skin during sleep. Each of these materials may include an element of elasticity as well. Certainly, other materials with similar properties may be used for the eye portions, 12 and 14.

Also, the two portions, 12 and 14, may be independent of one another (e.g., like lenses in a pair of glasses), providing adjustability between the two portions, or they may be a single, unitary design connected by similar material, as shown in FIG. 2. In either embodiment, pleats 24 may be used to provide greater space in the user's eye area, thereby applying minimum pressure on the user's eyeball during sleep.

Unique to the present mask 10 is the extended length of each eye portion, 12 and 14. Preferably, each eye portion, 12 and 14, extends from at or above a user's eyebrow to just below the cheekbone. Of course, the mask 10 could be made in various adult and child sizes (e.g., small, medium, large, etc.) to suit different sized users.

Referring now to FIGS. 3-5, a securing strap 18 can be seen connecting to an outer edge of each of the first and second eye portions, 12 and 14, to facilitate retention of the mask 10 in a desired position. The securing strap 18 may be an elastic material to provide a range of head sizes to which it may accommodate. Alternatively, the strap 18 may be adjustable in length using any number of adjustment mechanisms known in the art. A thin cord securing strap 18 is illustrated in FIG. 3, while a wider strap 19 is used in the embodiment of FIG. 5. The strap 19 of FIG. 5 may include VELCRO® (generically known as “hook-and-loop” material) to provide size adjustability.

The securing strap 18/19 may be permanently or temporarily secured to the mask 10 using any known methods. For example, the ends may be sewn directly into the eye portions, 12 and 14, or attached using grommets, clasps, Velcro®, or the like. As shown, the thin cord securing strap 18 has two connection points to spread the tension more evenly across the mask 10. The wider strap 19 may not require this type of attachment.

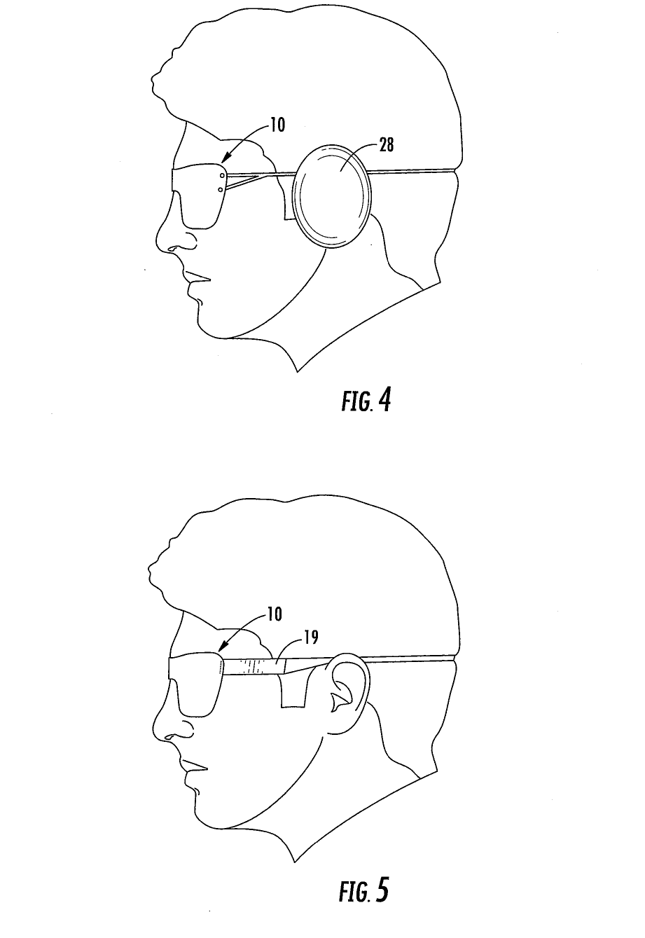

Additional features for the mask 10 include optional ear protectors 28, as shown in FIG. 4. The ear protectors 28 offer another level of sleep protection for the user to help facilitate a deep, uninterrupted sleep by blocking extraneous noise, which can be particularly useful for daytime sleepers. The ear protectors 28 are preferably attachable/detachable to the securing strap 18/19 so as to be positioned to cover a user's ears. The protectors 28 may attach under the securing strap 18/19 using snaps, buttons, Velcro, grommets, or the like. They may be made from the same or similar material as the eye portions, 12 and 14. Alternatively, other materials may be used which are able to provide the requisite comfort and sound-blocking properties.

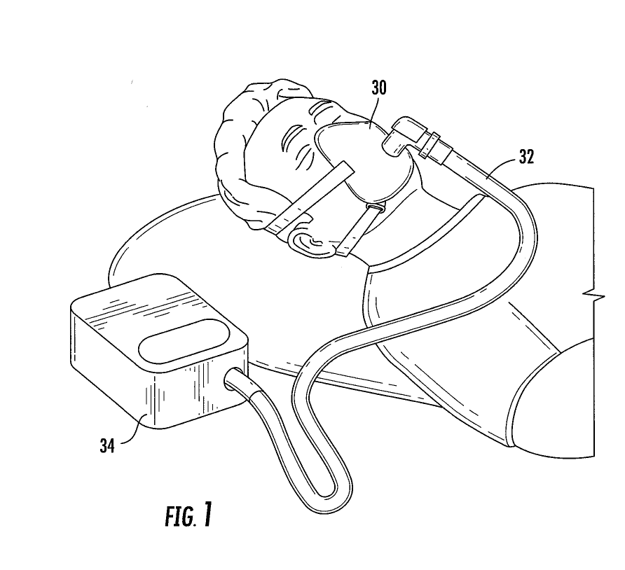

In use, the protective mask 10 is placed about the user's head with the eye portions, 12 and 14, properly aligned to be seated over the user's eye area. The securing strap 18/19 may then be adjusted in length for comfort. Referring to FIG. 1, an air-delivery mask 30 formed to fit over at least the user's nose (and in some cases the nose and mouth) and having tubing 32 connecting the air-delivery mask 30 to an air pump 34, as is used in most CPAP systems, is put into place. The air pump 34 may be turned on to start a flow of continuous pressure air to the air-delivery mask 30. The protective mask 10 may then be moved to cover the user's eye area. The protective mask 10 may overlap with the air-delivery mask 30, in which case the protective mask 10 should be positioned under the air-delivery mask 30. If the optional ear protectors 28 (FIG. 4) are used, these too may be attached and positioned over the user's ears and adjusted for. comfort.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. An eye-protection device for use with a CPAP machine and headgear, including an oxygen mask which covers a user's nose, the device comprising:
a first eye portion sized to extend from a person's eyebrow to an area below a person's cheekbone when in use;
a second eye portion connected to the first eye portion and sized to extend from a person's eyebrow to an area below a person's cheekbone when in use; and
a securing strap connected to each of the first and second eye portions for retaining the device on a user;
wherein the first eye portion and the second eye portion are comprised of a material having a soft porous inner surface layer and a non-porous outer surface layer.

2. The eye-protection device of claim 1, further comprising an edging along a periphery of the first eye portion and the second eye portion, wherein the edging is comprised of a soft porous material.

3. The eye-protection device of claim 1, wherein the securing strap has an adjustable length.

4. The eye-protection device of claim 3, wherein the securing strap comprises a hook-and-loop connection.

5. The eye-protection device of claim 1, further comprising a first expandable section in the first eye portion and a second expandable section in the second eye portion.

6. The eye-protection device of claim 5, wherein the first and second expandable sections comprise pleats formed in the material of the first and second eye portions.

7. The eye-protection device of claim 1, further comprising first and second ear protectors attached to the securing strap, positioned to cover a person's ears when in use.

8. The eye-protection device of claim 7, wherein the position of the first and second ear protectors is adjustable along a length of the securing strap.

9. The eye-protection device of claim 1, wherein the non-porous outer surface layer comprises a vinyl material.

10. The eye-protection device of claim 9, wherein the porous inner surface layer comprises a cotton material.

11. The eye-protection device of claim 1, wherein the material is stretchable.

12. The eye-protection device of claim 10, wherein the vinyl material is adhered to the entire surface of the porous material.

13. A system for treatment of a sleep disorder, the system comprising:
an pump having an outlet through which air is discharged at a constant pressure;
tubing having two open ends, a first end being connected to the outlet of the pump;
an air-delivery mask formed to fit over a user's nose and connected to a second end of the tubing, wherein during use a flow of constant pressure air is delivered from the pump, through the tubing to the mask; and
an eye mask for shielding the user's eyes from a flow of air escaping from the air-delivery mask, the eye mask comprising:
first and second eye portions comprised of a material having a soft porous inner surface layer and a non-porous outer surface layer; and
a securing strap connected to each of the first and second eye portions for retaining the eye mask on a user during sleep.

14. The system of claim 13, further comprising an edging along a periphery of the first eye portion and the second eye portion, wherein the edging is comprised of a soft porous material.

15. The system of claim 13, wherein the securing strap has an adjustable length.

16. The system of claim 15, wherein the securing strap comprises a hook-and-loop connection.

17. The system of claim 13, further comprising a first expandable section in the first eye portion and a second expandable section in the second eye portion.

18. The system of claim 17, wherein the first and second expandable sections comprise pleats formed in the material of the first and second eye portions.

19. The eye-protection device of claim 13, wherein the non-porous outer surface layer comprises a vinyl material.

20. The eye-protection device of claim 19, wherein the vinyl material is adhered to the entire surface of the porous material.
